SEQUENCE LISTING (31 sequences)

SEQ ID NO: 1 - protein, 49 aa, Human immunodeficiency virus
HTTWMEWDREINNYTSLIHSLIEESQNQQEKNEQELLELDKWASLWNWF

SEQ ID NO: 2 - protein, 37 aa, Human immunodeficiency virus
NYTSLIHSLIEESQNQQEKNEQELLELDKWASLWNWF

SEQ ID NO: 3 - protein, 37 aa, Human immunodeficiency virus
KYISLIHSLIEESQNQQEKNEQELLELDKWASLWNWF

SEQ ID NO: 4 - protein, 37 aa, Human immunodeficiency virus
HTTWMEWDREINNYTSLIHSLIEESQNQQEKNEQELL

SEQ ID NO: 5 - protein, 37 aa, Human immunodeficiency virus
HTTWMEWDREINKYISLIHSLIEESQNQQEKNEQELL

SEQ ID NO: 6 - protein, 39 aa, Human immunodeficiency virus
WQEWEQKITALLEQAQIQQEKNEYELQKLDKWASLWEWF

SEQ ID NO: 7 - protein, 345 aa, Human immunodeficiency virus
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 8 - protein, 49 aa, Human immunodeficiency virus; identical to SEQ ID NO: 1

SEQ ID NO: 9 - protein, 37 aa, Human immunodeficiency virus; identical to SEQ ID NO: 4

SEQ ID NO: 10 - protein, 34 aa, Human immunodeficiency virus
WMEWDREINNYTSLIHSLIEESQNQQEKNEQELL

SEQ ID NO: 11 - protein, 36 aa, Human immunodeficiency virus
YTSLIHSLIEESQNQQEKNEQELLELDKWASLWNWF

SEQ ID NO: 12 - skipped

SEQ ID NO: 13 - protein, 10 aa, Human immunodeficiency virus
QLLSGIVQQQ

SEQ ID NO: 14 - protein, 10 aa, Human immunodeficiency virus
QLLSDTVQQQ

SEQ ID NO: 15 - protein, 10 aa, Human immunodeficiency virus
QLLSGIEQQQ

SEQ ID NO: 16 - protein, 49 aa, Artificial Sequence
  Description of Artificial Sequence: Synthetic peptide
GGHTTWMEWDREINNYTSLIHSLIEESQNQQEKNEQELLGGHHHHHHGC

SEQ ID NO: 17 - protein, 84 aa, Artificial Sequence
  Description of Artificial Sequence: Synthetic peptide
HTTWMEWDREINNYTSLIHSLIEESQNQQEKNEQELLGGHTTWMEWDREINNYTSLIHSLIEESQNQQEKNEQELLGGHHHHHH

SEQ ID NO: 18 - protein, 126 aa, Artificial Sequence
  Description of Artificial Sequence: Synthetic polypeptide
  MOD_RES (38)..(40): Any amino acid
  MOD_RES (78)..(80): Any amino acid
  MOD_RES (118)..(120): Any amino acid
NYTSLIHSLIEESQNQQEKNEQELLELDKWASLWNWFXXXNYTSLIHSLIEESQNQQEKNEQELLELDKWASLWNWFXXXNYTSLIHSLIEESQNQQEKNEQELLELDKWASLWNWFXXXHHHHHH

SEQ ID NO: 19 - protein, 77 aa, Artificial Sequence
  Description of Artificial Sequence: Synthetic peptide
  MOD_RES (38)..(40): Any amino acid
NYTSLIHSLIEESQNQQEKNEQELLELDKWASLWNWFXXXHTTWMEWDREINKYISLIHSLIEESQNQQEKNEQELL

SEQ ID NO: 20 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 21 - protein, 12 aa, Artificial Sequence
  Description of Artificial Sequence: Synthetic peptide
LGGGGSGGGGSA

SEQ ID NO: 22 - protein, 13 aa, Artificial Sequence
  Description of Artificial Sequence: Synthetic peptide
AEAAAKEAAAKEA

SEQ ID NO: 23 - protein, 40 aa, Artificial Sequence
  Description of Artificial Sequence: Synthetic peptide
  MOD_RES (6)..(40): May or may not be present
GGGGSGGGGSGGGGSGGGGSGGGGSGGGGSGGGGSGGGGS

SEQ ID NO: 24 - protein, 6 aa, Artificial Sequence
  Description of Artificial Sequence: Synthetic peptide
HHHHHH

SEQ ID NO: 25 - protein, 40 aa, Human immunodeficiency virus
QLLSGIEQQQNNLLRAIEAQQHLLQLTVWGIKQLQARILA

SEQ ID NO: 26 - protein, 40 aa, Human immunodeficiency virus
QLLSDTVQQQNNLLRAIEAQQHLLQLTVWGIKQLQARILA

SEQ ID NO: 27 - protein, 40 aa, Human immunodeficiency virus
QLLSGIVQQQNNLLRAIEAQQHLLQLTVWGIKQLQARILA

SEQ ID NO: 28 - protein, 47 aa, Artificial Sequence
  Description of Artificial Sequence: Synthetic peptide
GGHTTWMEWDREINNYTSLIHSLIEESQNQQEKNEQELLGGHHHHHH

SEQ ID NO: 29 - protein, 47 aa, Artificial Sequence
  Description of Artificial Sequence: Synthetic peptide
GGHTTWMEWDREINKYISLIHSLIEESQNQQEKNEQELLGGHHHHHH

SEQ ID NO: 30 - protein, 47 aa, Artificial Sequence
  Description of Artificial Sequence: Synthetic peptide
GGHTTWMEWDREINKYTSLIHSLIEESQNQQEKNEQELLGGHHHHHH

SEQ ID NO: 31 - protein, 47 aa, Artificial Sequence
  Description of Artificial Sequence: Synthetic peptide
GGHTTWMEWDREINNYISLIHSLIEESQNQQEKNEQELLGGHHHHHH

(19) United States
(12) Patent Application Publication
(10) Pub. No.: US 2010/0029568 A1
(43) Pub. Date: Feb. 4, 2010

(54) TIGHTER-BINDING C-PEPTIDE INHIBITORS OF HIV-1 ENTRY

(75) Inventors: Kristen Kahle, Narbeth, PA (US); Suparna Paul, Clifton Heights, PA (US); H. Kirby Steger, Wallingford, PA (US); Michael J. Root, Philadelphia, PA (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/531,116

(22) PCT Filed: Mar. 12, 2008
(86) PCT No.: PCT/US08/56609

§ 371 Date: Sep. 14, 2009

Related U.S. Application Data
(60) Provisional application No. 60/906,421, filed on Mar. 12, 2007.

(51) Int. Cl. A61K 38/16; C07H 21/04; A61P 31/18

(52) U.S. Cl. 514/12; 536/23.72

(57) ABSTRACT

The invention provides compositions and methods for the treatment of HIV infection, inhibition against drug-resistant strains of HIV-1 and methods of enhancing the anti-HIV potency of peptide inhibitors against drug-resistant strains of HIV-1. In particular, oligomeric C-peptide inhibitors for inhibiting HIV entry into host cells are disclosed.

### CROSS REFERENCE TO RELATED APPLICATIONS

This application claims the benefit under 35 U.S.C. §119(e) of U.S. provisional application No. 60/906,421 filed Mar. 12, 2007, the contents of which are incorporated herein by reference in its entirety.

### GOVERNMENT SUPPORT

The invention was made with Government support under grant GM66682 awarded by the National Institutes of Health. The Government has certain rights in the invention.

### BACKGROUND OF THE INVENTION

Human immunodeficiency virus (HIV) is a retrovirus that causes acquired immunodeficiency syndrome (AIDS, a condition in humans in which the immune system begins to fail, leading to life-threatening opportunistic infections). HIV infection continues to be a major global health problem. There is currently no vaccine or cure for HIV or AIDS. Current anti-HIV therapies targeting reverse transcriptase and protease enzymes suffer from high cost, a high probability of engendering resistance and adverse side effects following prolonged use. Thus, there is still the need to develop new antiviral strategies with more potent compounds and/or novel antiviral targets.

The characterization of the HIV cell-fusion mechanism and the initial mapping of the interactions of the associated proteins involved in this process has provided an opportunity to identify and take advantage of chemokine co-receptors as new antiviral targets. The HIV fusogenic particle consists of the virus-derived gp120, gp41, cell-derived CD4 and chemokine co-receptors, all of which must interact in a concerted fashion to allow entry of the virus into the cell. The structural analysis of these components has resulted in the identification of a number of new antiviral fusion targets that are distinct from the gp120:CD4 binding. Three types of fusogenic particle antagonists have emerged: (1) ribozyme-based gene therapy targeting the chemokine co-receptors; (2) peptide-based antagonists targeting either domains of gp41 or the chemokine co-receptors; and (3) small molecule inhibitors targeting the virus:co-receptor interaction.

Among the fusogenic particle antagonists, peptide-based antagonists have shown great promise towards advancing the development of new anti-HIV therapeutics. C-peptides are the collective name for polypeptide drugs derived from the C-terminal region of the extracellular domain (ectodomain) of the HIV-1 transmembrane glycoprotein gp41. Together with the surface glycoprotein gp120, gp41 mediates the entry of HIV-1 through fusion of viral and cellular membranes. This process involves a series of coordinated structural changes initiated by the interaction of gp120 with target cell CD4 and culminating with the collapse of the gp41 ectodomain into a trimer-of-hairpins structure. The thermostable core of this final conformation is a bundle of six α-helices formed by the association of the HR1 and HR2 heptad repeat regions from three gp41 ectodomains. C-peptides, derived from the HR2 region, block formation of the gp41 trimer-of-hairpins by binding to HR1 regions prior to fusion, thereby inhibiting viral entry.

Currently, there is only one peptide-based antagonist approved for treatment of HIV-infection in human. The C-peptide T20 (enfuvirtide—Hoffmann-La Roche & Trimeris; U.S. Pat. No. 5,464,933) is the first HIV-1 entry inhibitor approved by the FDA for treatment of patients suffering from AIDS. It is currently utilized heavily in salvage therapy for patients who have failed treatment with other, more conventional medications (reverse transcriptase and protease inhibitors). However, a major problem with the clinical use of T20 is the rapid emergence of resistance mutations. Hence, there is also a pressing need for new anti-HIV strategies with more potent compounds and/or novel antiviral targets against the common and resistant strains of HIV-1 as well as new therapeutic strategies aimed at preventing the development of HIV resistant stains during HIV treatment regimes.

### SUMMARY OF THE INVENTION

Embodiments of the present invention are based on the discovery that homo-dimers of C37 or homo-dimers of T20 C-peptide inhibitors of HIV cell fusion are more efficacious than monomeric C-peptides or T20 in inhibiting HIV entry of the HIV that have become resistant to the monomeric C37 or T20. Homo-dimers of C37 have two, identical monomeric C37 peptide inhibitors and homo-dimers of T20 two, identical monomeric T20 peptide inhibitors.

In one embodiment, a composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined by a molecular linker is provided. The peptide fragment used in the composition of this embodiment is selected from the group of peptide fragments consisting of the amino acid sequences:

### A

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;
and

(SEQ. ID. No. 5)

HTTWMEWDREINKYISLIHSLIEESQNQQEKNEQELL.

Peptides with mutations in the NYT of SED. ID. No. 1 such as KYT, KYI, and NYI are included in the invention.

The physical joining of a plurality of peptides by a molecular linker results in an oligomer of peptides. The composition can comprise a oligomeric peptide that is a dimer of two peptides, a trimer of three peptides, a tetramer of four peptides, or a pentamer of five peptides. In a preferred embodiment, the oligomeric peptide is a dimer of two peptides and/or a trimer of three peptides. In one embodiment, the oligomeric peptide is a homo-oligomeric peptide, comprising identical peptides according to the invention disclosed herein. Hetero-oligomeric peptides comprising different peptides, fragments, and/or variants thereof are also contemplated.

In one embodiment, the molecular linker that joins the peptides to form an oligomeric peptide can be a peptide linker molecule or a chemical linker. The peptide linker molecule can comprise at least 2, 3, 4, 5, 6, 7, 8, 9, or 10 amino acids residues.

In one embodiment, a method is provided to enhance the anti-HIV potency of a given HIV C-peptide inhibitor, the method comprising physically joining a plurality of molecules of C-peptide inhibitors by a molecular linker. The C-peptide inhibitor of this embodiment is selected from a group of C-peptides consisting of the amino acid sequences:

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;

(SEQ. ID. No. 5)

HTTWMEWDREINKYISLIHSLIEESQNQQEKNEQELL;
and

(SEQ. ID. No. 6)

WQEWEQKITALLEQAQIQQEKNEYELQKLDKWASLWEWF.

The physical joining of a plurality of peptides by a molecular linker results in an oligomer of C-peptide inhibitors. The oligomeric C-peptide inhibitor can be a dimer of two C-peptides, a trimer of three C-peptides, a tetramer of four C-peptides, or a pentamer of five C-peptides. In a preferred embodiment, the oligomeric C-peptide inhibitor is a dimer of two C-peptides and/or a trimer of three C-peptides. In one embodiment, the oligomeric C-peptide inhibitor is a homo-oligomeric C-peptide inhibitor, comprising identical C-peptides as described herein. Hetero-oligomeric C-peptide inhibitors comprising different C-peptides, fragments, and/or variants thereof are also contemplated.

In one embodiment, the molecular linker that joins the C-peptide inhibitors to form an oligomeric C-peptide inhibitor can be a peptide linker molecule or a chemical linker. The peptide linker molecule can comprise at least 2, 3, 4, 5, 6, 7, 8, 9, or 10 amino acids residues.

In one embodiment, a pharmaceutical composition is provided, comprising a composition with anti-HIV activity comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined by a molecular linker, and a pharmaceutically acceptable carrier. In this embodiment, the peptide fragment used in the composition is selected from the group of peptide fragments consisting of the amino acid sequences:

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;
and

(SEQ. ID. No. 5)

HTTWMEWDREINKYISLIHSLIEESQNQQEKNEQELL.

In one embodiment, an isolated nucleic acid is provided that encodes a protein comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined by a peptide linker molecule. The protein encoded by the isolated nucleic acid can be a dimer of two peptides, a trimer of three peptides, a tetramer of four peptides, or a pentamer of five peptides. In a preferred embodiment, the protein is a dimer of two peptides and/or a trimer of three peptides. In one embodiment, the encoded protein is a homo-oligomeric peptide, comprising identical peptides as described herein. Hetero-oligomeric peptide comprise different peptides, fragments, and/or variant thereof are also contemplated. The identical or different peptides in the encoded protein can be separated by spacer amino acids such as glycine, tyrosine, cysteine, lysine, proline, glutamic and aspartic acid. In one embodiment, the spacer amino acid length between peptides in the protein is at least 2, 3, 4, 5, 6, 7, 8, 9, or 10 amino acids residues.

In one embodiment, a method is provided for treating HIV infection that is resistant to anti-HIV therapy in a subject, the method comprising administering to the subject in need thereof, an effective amount of a composition with anti-HIV activity, the composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the peptides are physically joined by a molecular linker.

In one embodiment, a method is provided for preventing the development of HIV resistance to anti-HIV therapy in a subject, the method comprising administering to the subject in need thereof an effective amount of a composition with anti-HIV activity, the composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the peptides are physically joined by a molecular linker, in combination with anti-viral HIV therapy.

Embodiments of the invention disclosed herein can be implemented with other anti-viral HIV therapies include HIV protease inhibitors, the HAART and HIV integrase inhibitors.

### BRIEF DESCRIPTION OF THE DRAWINGS

FIG. 1A shows a schematic diagram showing the design of C-peptides with enhanced binding affinity.

FIG. 1B shows the axial view of the six-helix bundle that forms the core of the gp41 trimer-of-hairpins. The model is based on the crystal structure of N36 and C34 peptides (underlined residues in WT and C37 sequences in FIG. 1A).

FIG. 1C shows the lateral view of the six-helix bundle that forms the core of the gp41 trimer-of-hairpins. The model is based on the crystal structure of N36 and C34 peptides (underlined residues in WT and C37 sequences in FIG. 1A).

FIG. 2A shows the inhibition of T20- and C37-resistant HIV-1 by wild type C37 peptide.

FIG. 2B shows the inhibition of T20- and C37-resistant HIV-1 by wild type C37_KYI peptide. Viruses pseudotyped with either wild type Env (closed squares) or V549E mutant Env (Rest, closed circles) from strain HXB2 were utilized to infect HOS-CD4-Les target cells.

FIG. 2C shows the inhibition of T20- and C37-resistant HIV-1 by wild type (C37_GC)₂ peptide.

### DETAILED DESCRIPTION OF THE INVENTION

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless otherwise stated, the compositions and methods described herein are made or used using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Virology Methods Manual, First Edition (Hillar O. Kangro, ed., Academic Press, 1996), Culture of Animal Cells: A Manual of Basic Technique by R. Ian Freshney, Publisher: Wiley-Liss; 5th edition (2005), Animal Cell Culture Methods (Methods in Cell Biology, Vol 57, Jennie P. Mather and David Barnes editors, Academic Press, 1st edition, 1998) which are all incorporated by reference herein in their entireties.

### DEFINITIONS

The one- and three-letter abbreviations used herein for the various common amino acids are as recommended in Pure Appl. Chem. 31, 639-645 (1972) and 40, 277-290 (1974) and comply with 37 CFR §1.822 (55 FR 18245, May 1, 1990). The abbreviations represent L-amino acids unless otherwise designated as D- or D,L-. Certain amino acids, both natural and non-natural, are achiral, e.g. glycine. All peptide sequences are presented with the N-terminal amino acid on the left and the C-terminal amino acid on the right.

“Amino acid” is used herein to refer to a chemical compound with the general formula: NH₂—CRH—COOH, where R, the side chain, is H or an organic group. Where R is organic, R can vary and is either polar or nonpolar (i.e., hydrophobic). The amino acids of this invention can be naturally occurring or synthetic (often referred to as non proteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted. The twenty common amino acids known in the art are the preferred amino acids used in the synthesis of the peptides described herein.

As used herein, the term “anti-HIV activity” refers to activities that inhibit, block, impede, prevent or stop the entry of HIV virus into host cells, and thus inhibit the replication and multiplication of the HIV virus in host cells, and the spread of the HIV virus in a subject infected with the HIV virus. Anti-HIV activity also refers to the inhibition of fusion of the viral and host cellular membranes.

The terms “polypeptide” and “peptide” are used interchangeably herein to refer to a polymer of amino acids. These terms do not connote a specific length of a polymer of amino acids. Thus, for example, the term includes oligomeric peptides, made up of two or more physically linked peptides, whether produced using recombinant techniques, chemical or enzymatic synthesis, or naturally occurring. This term also includes polypeptides that have been modified or derivatized, such as by glycosylation, acetylation, phosphorylation, and the like.

As used herein, a C-peptide is a peptide comprising the amino acid sequence derived from the C-terminal extracellular domain (ectodomain) heptad repeat region 2 (HR2) region of the HIV-1 transmembrane glycoprotein gp41 protein (SEQ. ID. No. 7, Genbank Accession No. NP_{—}579895), comprising the amino acids 114-162 (SEQ. ID. No. 8). This C-terminal heptad repeat region 2 (HR2) of gp41 is the source of several widely known C-peptides: C37 corresponding to amino acids 114-150 (SEQ. ID. No. 9) (Root, M., et. al., 2001, Science, 291: 884), C34 corresponding to amino acids 117-150 (SEQ. ID. No. 10) (Chan, C., et. al., 1997, Cell 89: 263; Nameki, D., et. al., 2005, J. Virol., 79:764), and T20 corresponding to amino acids 127-162 (SEQ. ID. No. 11) (U.S. Pat. No. 5,464,933) are derived from this C-terminal domain. A C-peptide can comprise all 49 amino acids, or smaller fragments thereof, such as C-peptides with 37, 34, or 36 amino acids.

As used herein, the term “fragment” refers to an amino acid sequence which has less than the 49 amino acids and more than 10 amino acids corresponding to the amino acid sequence of HR2 region of gp41 (amino acid 114-162). These C-peptides and “fragments” have inhibitory activity against HIV viral entry into host cells. Methods known in the art and described herein can be used for assessing inhibition of virus entry into host cell activity of C-peptides.

As used herein, the term “inhibit” or “inhibition” means the reduction and/or prevention of HIV viruses (multiple strains) infecting new host cells and the spread of the infection within a human subject. The inhibition can be determined by various methods known in the art, such as in vitro experimentation of HIV viral entry into cultured host cells or a measure of the number of CD4⁺ T cell circulating in an HIV positive human subject. The strains of HIV include drug-resistant and non-drug resistant strains, and include strain HIV-1 and HIV-2 and the variant strains of HIV-1 and HIV-2. “Inhibition” includes slowing the rate of virus infectivity. When HIV infection is “inhibited”, the rate of infectivity is reduced by at least about 20%, about 30%, about 40%, about 50%, about 60%, about 70%, about 80%, about 90% or by as much as 100% compared to the absence of an anti-HIV composition as described herein. Cell fusion inhibition assays are fully described in U.S. Pat. Nos. 5,464,933 and 6,656,906 which are hereby incorporated by reference.

As used herein, the term “C-peptide inhibitor” is a C-peptide as described supra that inhibits HIV viral entry. The terms “C-peptides” used herein and “C-peptide inhibitors are used interchangeably.

The terms “treat” and “treatment” refer to the therapeutic treatment wherein the subject has been diagnosed as having been infected by the HIV virus. The terms “treat” and “treatment” refer to preventing or slowing the infection and destruction of healthy CD4+ T cells in such a subject. It also refers to the prevention and slowing the onset of symptoms of the acquired immunodeficiency disease such as extreme low CD4+ T cell count and repeated infections by opportunistic pathogens such as Mycobacteria sp., Pneumopcystis carinii, Pneumopcystis cryptococcus, and so on. Beneficial or desired clinical results include, but are not limited to, an increase in absolute naïve CD4+ T-cell count (range 10-3520), an increase in the percentage of CD4+ T-cell over total circulating immune cells (range 1-50%), and/or an increase in CD4+ T-cell count as a percentage of normal CD4+ T-cell count in an uninfected subject (range 1-161%). “Treatment” can also mean prolonging survival of the infected subject as compared to expected survival if the subject did not receiving any HIV targeted treatment.

The efficacy of treatment can be assessed by monitoring the viral load and CD4+ T cell count in the blood of an infected subject. There should be greater than or equal to one log reduction in viral load, preferably to less than 10,000 copies/ml HIV-RNA within 2-4 weeks after the commencement of treatment. If <0.5 log reduction in viral load, or HIV-RNA stays above 100,000, then the treatment should be adjusted by either adding or switching drugs. Viral load measurement should be repeated every 4-6 months if the patient is clinically stable. If viral load returns to 0.3-0.5 log of pre-treatment levels, then the therapy is no longer working and should be changed. Within 2-4 weeks of starting treatment, CD4+ T-cell count should be increased by at least 30 cells/mm³. If this is not achieved, then the therapy should be changed. Monitoring of the CD4+ T-cell counts should be obtained every 3-6 months during periods of clinical stability, and more frequently should symptomatic disease occur. If CD4+ T-cell count drops to baseline (or below 50% of increase from pre-treatment), then the therapy should be changed.

A “therapeutically effective amount” means that amount necessary for reducing the HIV virus entry into host cells in the absence of additional anti-HIV therapy. The amount should lead to a reduction in viral load and an increase in the CD4+ T cell count in the HIV-infected subject. The reduction in viral load is at least about 30%, about 40%, about 50%, about 60%, about 70%, about 80%, about 90%, about 100%, or more and all the percentages between, compared to in the absence of any anti-HIV composition described herein. The CD4+ T-cell count should be at least about 30%, about 40%, about 50%, about 60%, about 70%, about 80%, about 90%, about 100%, or more, and all the percentages in between, of a healthy non-HIV infected CD4+ T cell count. It is preferred generally that a maximum dose be used, that is, the highest safe dose according to sound medical judgment. It will be understood by those of ordinary skill in the art; however, that a lower dose or tolerable dose may be administered for medical reasons, psychological reasons, when another anti-HIV drugs are simultaneously administered or for another reason, within the discretion of the treating clinician.

As used herein, the term “variant forms of C-peptide” refers to C-peptides (or to nucleic acid sequences encoding them) modified at one or more amino acid, nucleotide base pairs, codons, introns, or exons, respectively, that retain at least 80% of the biological activity and cellular function of wild-type C-peptide as determined by its inhibition activity of HIV entry into host cells. Thus, a variant C-peptide sequence is slightly different from that prescribed by the C-terminal HR2 domain of the gp41 gene (SEQ. ID. No. 12) (Genbank Accession No. BD407105, NC_{—}001802, AJ293865). There are one or more amino acid mutations with conserved or non-conserved amino acid residues in a variant C-peptide. For example, the amino acid serine can be substituted for threonine and the amino acid aspartate can be substituted for glutamate. A variant C-peptide may differ in amino acid sequence by one or more substitutions. Among preferred variants are those that vary from a reference polypeptide by conservative amino acid substitutions. Such substitutions are those that substitute a given amino acid by another amino acid of like characteristics. Conservative substitutions are the replacements, one for another, among the aliphatic amino acids Ala, Val, Leu and Ile; interchange of the hydroxyl residues Ser and Thr; exchange of the acidic residues Asp and Glu; substitution between amide residues Asn and Gln; exchange of the basic residues Lys and Arg; and replacements among aromatic residues Phe and Tyr. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionin and valine and f) phenylalanine, tyrosine and tryptophan. Most highly preferred are variant C-peptides which retain the same biological function and activity as the parent C-peptide from which it varies, which is inhibition of HIV viral entry into host cells.

A C-peptide with a N→K mutation at Asp 126 of the gp41 protein (SEQ. ID. No. 7) (Asp637 in the gp160 precursor glycoprotein, SEQ. ID. No. 20) and or a T→I mutation at Thr128 of gp41 protein (corresponding to Thr639 of gp160) are considered variant C-peptides. For example, KYISLIHSLIEESQNQQEKNEQELLELDKWASLWNWF (SEQ. ID. No. 3) is a variant of NYTSLIHSLIEESQNQQEKNEQELLELDKWASLWNWF (SEQ. ID. No. 2), and HTTWMEWDREINKYISLIHSLIEESQNQQEKNEQELL (SEQ. ID. No. 5) is a variant of HTTWMEWDREINNYTSLIHSLIEESQNQQEKNEQELL (SEQ. ID. No. 4). Other variant C-peptides of the HR2 region of gp41 are described in US Patent Application 2006/0247416 which is hereby incorporated by reference.

Variant C-peptides as the term “variant” is used herein, have comparable or greater HIV entry inhibition activity than the parent C-peptide. A variant C-peptide will have at least 80%, at least 90%, at least 100%, at least 110%, at least 120%, at least 130%, at least 140%, or at least 150% of the HIV entry inhibition activity of the parent C-peptide. Variants can be produced by a number of means including methods such as, for example, error-prone PCR, shuffling, oligonucleotide-directed mutagenesis, recursive ensemble mutagenesis, exponential ensemble mutagenesis, site-specific mutagenesis, gene reassembly, GSSM and any combination thereof. Methods known in the art and described herein can be used for assessing inhibition of virus entry into host cell by C-peptides.

As used herein, the term “an oligomer,” when used in reference to C-peptides, refers to a complex made up of a finite number of building blocks of monomer C-peptides. Oligomerization is the process of converting the monomers of C-peptides into a complex of C-peptides.

As used herein, a peptide linker is a short sequence of amino acids that is not part of the sequence of either of the two peptides being joined. A peptide linker is attached on its amino-terminal end to one polypeptide or polypeptide domain and on its carboxyl-terminal end to another polypeptide or polypeptide domain. Examples of useful linker peptides include, but are not limited to, glycine polymers ((G)n) including glycine-serine and glycine-alanine polymers (e.g., a (Gly₄Ser)n repeat where n=1-8, preferably, n=3, 4, 5, or 6). The peptide linker can be a flexible linker, in that the peptide sequence does not adopt any secondary structures known in proteins, eg. alpha helices. Such flexible linkers are predominantly made of non-charged, apolar amino acid residues and are hydrophobic. Secondary protein structures can be determined by methods known in the art, for example, circular dichroism. A example of a flexible peptide linker is LGGGGSGGGGSA (SEQ. ID. No. 21). Alternately, the peptide linker can take the form a monomeric hydrophilic α-helix, for example, AEAAAKEAAAKEA (SEQ. ID. No. 22).

The term “vector”, as used herein in reference to constructs encoding, e.g., C-peptides, refers to a nucleic acid construct comprising the complete or partial coding sequence of the HR2 region of the HIV-1 transmembrane glycoprotein gp41 protein, amino acids 114-162 (SEQ. ID. No. 12) (Genbank Accession No. BD407105, NC_{—}001802, AJ293865), wherein the nucleic acid construct is designed for delivery to a host cell, transfer between different host cells, or for the expression of C-peptides, oligomeric C-peptides, or C-peptide variants thereof, in cells. The nucleic acid construct can have several copies of the coding sequence of the HR2 region arranged in tandem so as to express a polypeptide with repeated HR2 regions. As used herein, a vector can be viral or non-viral.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Embodiments of the present invention are based on the discovery that oligomers of the known HIV C-peptide inhibitors are equally as effective at inhibiting HIV entry as the monomeric HIV C-peptide inhibitors, where the IC₅₀ inhibition of cell entry determined for the oligomer is at the nanomolar range. More surprising is that these oligomeric C-peptide inhibitors are more effective against C-peptide-resistant strains of HIV. These resistant strains of HIV have become resistant to the monomeric parent C-peptide inhibitor such that the monomeric parent C-peptides are no longer effective in inhibiting HIV cell fusion and subsequent cell entry. The IC₅₀ inhibition values of resistant strain HIV cell entry for these oligomeric C-peptide inhibitors are also in the nanomolar range.

C-peptides are the collective name for peptide drugs derived from the C-terminal region of the extracellular domain (ectodomain) of the HIV-1 transmembrane glycoprotein gp41 (SEQ. ID. No. 7, Genbank Accession No. NP_{—}579895), mainly from the heptad repeat (HR2) region. Together with the surface glycoprotein gp120, gp41 mediates the entry of HIV-1 through fusion of viral and host cell cellular membranes. This process involves a series of coordinated structural changes initiated by the interaction of gp120 with target cell surface receptor CD4 and culminating with the collapse of the gp41 ectodomain into a trimer-of-hairpins structure. The thermostable core of this final conformation is a bundle of six α-helices formed by the association of the HR1 and HR2 heptad repeat regions from three gp41 ectodomains. The HR2 interacts with HR1 and is involved the formation of the trimer-of-hairpins structure. C-peptides, derived from the HR2 region, block the formation of the gp41 trimer-of-hairpins by binding to HR1 regions prior to membrane fusion, thereby inhibiting viral entry.

The C-peptide, T20, (enfuvirtide—Hoffmann-La Roche & Trimeris; U.S. Pat. No. 5,464,933) is the first HIV-1 entry inhibitor approved by the FDA for treatment of patients suffering from AIDS. It has a peptide sequence of YTSLIHSLIEESQNQQEKNEQELLELDKWASLWNWF (SEQ. ID. No. 11). Other known C-peptides that have been shown to be effective in blocking HIV-1 entry include C37 with a sequence of HTTWMEWDREINNYTSLIHSLIEESQNQQEKNEQELL (SEQ. ID. No. 9) (Root, M., et. al., 2001, Science 291: 884) and C34 with a sequence of WMEWDREINNYTSLIHSLIEESQNQQEKNEQELL (SEQ. ID. No. 10) (Chan, D., et. al., 1997, Cell 89:263-73; Malashkevich, V. N., et. al., 1998, Proc. Natl. Acad. Sci. USA 95: 9134-9). T20 is currently utilized heavily in salvage therapy for patients who have failed treatment with other, more conventional medications (reverse transcriptase inhibitors and protease inhibitors). T20 is very potent against HIV entry (IC₅₀˜1 nm). However a major problem with the clinical use of T20 is the rapid emergence of resistance mutations, where the IC₅₀ then becomes >900 nm. Attempts to produce C-peptide inhibitors properties similar to T20, such as T1249 by Trimeris Inc., have not been successful in clinical trials.

C-peptides inhibit gp41 in a kinetic window between the CD4-gp120 interaction and trimer-of-hairpins formation. As a consequence, their potency is not only dependent on binding affinity, but is also influenced by kinetic parameters such as the rate of association of C-peptides with gp41 and the lifetime of the sensitive intermediate state. These kinetic parameters tend to limit T20 and C37 potency to the low nanomolar range (variable depending on viral strain and infectable target cells). Tighter binding variants of T20 and C37 tend to inhibit wild type virus with the same nanomolar potencies.

Resistance to C-peptides develops through at least two different mechanisms. The first is straightforward and much more commonly observed: resistant viruses tend to accumulate mutations in the gp41 HR1 region, especially in the sequence between amino acids 543 and 552-QLLSGIVQQQ (SEQ. ID. No. 13) in HXB2 sequence that substantially reduce T20 and C37 binding affinity. Two commonly observed resistant profiles are QLLSDTVQQQ (SEQ. ID. No. 14) and QLLSGIEQQQ (SEQ. ID. No. 15), where the negative charge of the introduced Asp or Glu appears to substantially disrupt T20 and C37 binding. The second resistance profile, observed less frequently, involves mutations in the HR2 region of gp41. The mechanism behind this profile has not been fully ascertained, but the mutations in the HR2 region involving a conserved glycosylation site formed by Asn637 and Thr639 decrease the lifetime of the gp41 intermediate states, thereby reducing the amount of time C-peptides have to bind to gp41.

The present invention is related to compositions and methods for inhibiting HIV viral activity, both the HIV-therapy resistant viral strain and the non-resistant viral strains, and is also related to the preventing the development of the HIV-therapy resistant viral strain. In particular, the resistant viral strains are resistant to known C-peptide-based HIV therapy such as T20, C37, C34 and combinations thereof.

Encompassed in the present invention are strategies and sequences of C-peptide inhibitors that maintain their potency against common resistant strains of HIV-1. The described inhibitors are compositions of matter based on novel strategies to produce HIV-1 entry inhibitors that overcome common mechanisms of HIV resistance. Specifically, they encompass oligomeric C-peptides such as C37 and T20, which are established peptide inhibitors of HIV-1 entry as monomers. Oligomeric C-peptides also encompass oligomers of variant forms of C-peptides as described herein. Monomeric C-peptides are linked together physically to form an oligomeric C-peptide. As a monomer, C-peptides have been shown to have HIV entry inhibition activity. For example, T20 (enfuvirtide), the only FDA approved HIV-1 entry inhibitor in clinical use, has an IC50 of ˜1 nm. The oligomeric C-peptides described herein are just as potent (IC50˜1 nM) as the original monomeric C37 and T20 peptides against wild type HIV-1. More importantly, these oligomeric C-peptides retain this nanomolar potency in the setting of common viral escape mutations that confer resistance to the original monomeric C peptide inhibitors.

As used herein, C-peptides and C-peptide inhibitors are used interchangeably and both terms refer to C-peptides that can inhibit the entry of the HIV virus into the host cell. Methods of assaying HIV entry are described herein. The in vivo effects of such inhibition of HIV entry can be determined by a reduction in viral load and an increase in CD4+ T-cell count in the circulating peripheral blood of a subject infected with HIV. Methods of monitoring viral load and CD4+ T-cell count are well known in the art.

In one embodiment, the invention encompasses using existing and established C-peptide inhibitors with a few modifications. The modifications outlined in this specification are oligomerization and simple point mutations that do not substantially change the original C-peptide's chemical properties and would likely have only minimal effects on pharmacokinetic properties.

In one embodiment, the invention provides a composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined by a molecular linker. The physical linking of a plurality of peptides produces a complex that is an oligomeric peptide.

In one embodiment, peptide fragments or variants that inhibit HIV viral entry of the sequence HTTWMEWDREINNYTSLIHSLIEESQNQQEKNEQELLELDKWASLWNWF are used for the preparation of oligomeric peptide compositions. The peptide fragment or variant sequences have at least 75% identity with the sequence as herein disclosed.

In one embodiment, the peptides have at least 85% identity, more preferably at least 90% identity, even more preferably at least 95% identity, still more preferably at least 97% identity, and most preferably at least 99% identity with the amino acid sequences illustrated herein.

In a preferred embodiment, peptide fragment or variant is selected from the group of peptide fragments consisting of the amino acid sequences:

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;
and

(SEQ. ID. No. 5)

HTTWMEWDREINKYISLIHSLIEESQNQQEKNEQELL.

In one embodiment of the invention, two or more peptides are linked by a linker molecule. Preferably the linker molecule is a peptide linker. In one embodiment, the composition with anti-HIV activity comprises a plurality of peptides that are linked. The composition can have, for example, 2, 3, 4, 5, 6, 7, 8, 9, or 10 peptides linked together as an oligomeric peptide. In a preferred embodiment, the composition with anti-HIV activity comprises a dimer of two peptides, a trimer of three peptides, a tetramer of four peptides, or a pentamer of five peptides. In one embodiment, the composition with anti-HIV activity comprises a dimer of two peptides and/or a trimer of three peptides.

Encompassed are compositions of oligomeric polypeptides which comprise identical peptides according to the invention, herein referred to as homo-oligomeric peptides, as well as polypeptides comprising different-peptides, referred to as hetero-oligomeric peptides. In one embodiment, the composition with anti-HIV activity comprises a mixture of dimeric, trimeric, tetrameric, and pentameric peptides. In one embodiment, the mixture can also include the monomeric peptide along with oligomeric peptide. It is contemplated that all possible combinations of monomeric, dimeric, trimeric, tetrameric, and pentameric peptides, and homo-oligomeric peptides as well as hetero-oligomeric peptides can be included in the compositions described herein.

In one embodiment, the molecular linker used for forming the oligomeric polypeptides is a peptide linker molecule. In one embodiment, the peptide linking molecule comprises at least one amino acid residue which links at least two peptides according to the invention. The peptide linker comprises at least 2, 3, 4, 5, 6, 7, 8, 9, or 10 amino acids residues. The peptide linking molecule can couple polypeptides or proteins covalently or non-covalently. Typical amino acid residues used for linking are glycine, tyrosine, cysteine, lysine, glutamic and aspartic acid, or the like. A peptide linker is attached on its amino-terminal end to one peptide, polypeptide or polypeptide domain (e.g., a C-peptide) and on its carboxyl-terminal end to another peptide, polypeptide or polypeptide domain (again, e.g., a C-peptide). Examples of useful linker peptides include, but are not limited to, glycine polymers ((G)n) including glycine-serine and glycine-alanine polymers (e.g., a (Gly₄Ser)n repeat where n=1-8, preferably, n=3, 4, 5, or 6). Other examples of peptide linker molecules are described in U.S. Pat. No. 5,856,456 and is hereby incorporated by reference.

In another embodiment, the molecular linker is a chemical linker such as linkages by disulfide bonds between cysteine amino acid residues or by chemical bridges formed by amine crosslinkers, for example, glutaraldehyde, bis(imido ester), bis(succinimidyl esters), diisocyanates and diacid chlorides. Extensive data on chemical crosslinking agents can be found at Invitrogen's Molecular Probe under section 5.2.

In one embodiment, the oligomeric peptide can be made by linking individual isolated peptides. The individual peptides can be made by chemical methods known in the art or by recombinant methods also known in the art. For recombinant methods, the DNA coding sequence of a peptide can be made by amplification using the polymerase chain reaction (PCR) with the complete HR2 region of the HIV-1 transmembrane glycoprotein gp41 protein, amino acids 114-162 (SEQ. ID. No. 12) (Genbank Accession No. BD407105, NC_{—}001802, AJ293865) as a template for the PCR reaction. Specially designed PCR primers that incorporate restriction enzyme digestion sites and/or extra spacer or tag amino acid residues can be used to facilitate DNA ligation, recombinant protein expression, and protein purification. In order to facilitate linking of the peptides together, additional amino acid residues can be added, by way of the DNA coding sequence, to the peptides. For example, the thiol-group containing amino acid cysteine and the amine-group containing amino acid lysine can be added. The thiol-group and the amine group provide reactive groups useful for crosslinking reactions. In one embodiment, the additional amino acids are added at the ends of the peptides. The extra amino acids can be engineered into the coding sequence using standard recombinant molecular biology methods that are known in the art. In addition, extra amino acids that constitute a tag can be added to facilitate peptide expression and purifications. Examples of such tags include the thioredoxin first 105 amino acids, the tandem six histidine-tag, HA-tag, and the flag-tag. An example of such a peptide with terminally added cysteine groups and histidine (6×) purification tag is GGHTTWMEWDREINNYTSLIHSLIEESQNQQEKNEQELLGGHHHHHHGC (SEQ. ID. No. 16).

The DNA coding sequences of the different individual peptides can be ligated into expression vectors which are then transfected into appropriate expression host cells and induced to express the recombinant peptide. Subsequently, the expressed recombinant peptide can be purified and then used in cross-linking to form the dimeric, trimer, tetrameric, or pentameric oligomeric peptide compositions described herein by methods known in the art.

In the instance where the peptide contains no available reactive thiol-group for chemical cross-linking, several methods are available for introducing thiol-groups into proteins and peptides, including but not limited to the reduction of intrinsic disulfides, as well as the conversion of amine or carboxylic acid groups to thiol group. Such methods are known to one skilled in the art and there are many commercial kits for that purpose, such as from Molecular Probes division of Invitrogen Inc. and Pierce Biotechnology.

In another embodiment, the oligomeric peptide can be made by recombinant methods without the need for linking individual isolated peptides by chemical cross linking. Recombinant methods can be use to synthesize a single coding DNA sequence that comprises the several coding sequences of a peptide. For example, two and up to five peptide coding sequences are ligated in tandem. Additional amino acid coding sequences, coding for 2-10 amino acids, can be added between each pair of adjoining peptides as spacer sequences. When the single coding DNA is transcribed and translated, the expressed polypeptide can contain tandem repeats of peptides, each separated by 2-10 extra amino acids. Typical amino acid residues used for spacing sequences are glycine, tyrosine, cysteine, lysine, proline, glutamic and aspartic acid, or the like. In a preferred embodiment, the oligomeric peptide is expressed in an amino-carboxyl-amino-carboxyl tandem configuration. Similarly, the oligomeric peptide synthesized can include a tag amino acid sequence for facilitating oligomeric peptide expression, identification and purifications. Such recombinant methods are well known to one skilled in the art.

In one embodiment, the complex of oligomeric peptides can be modified by NH₂-terminal acylation, e.g., acetylation, or thioglycolic acid amidation, by terminal-carboxylamidation, e.g., with ammonia, methylamine, and the like terminal modifications that are known in the art. Terminal modifications are useful to reduce susceptibility by proteinase digestion, and therefore serve to prolong half life of the polypeptides in solutions, particularly biological fluids where proteases may be present.

In one embodiment, the invention also provides a method of enhancing the anti-HIV potency of a given HIV C-peptide inhibitor, the method comprising physically joining a plurality of C-peptide inhibitors by a molecular linker. In another embodiment, fragments and/or variant forms of a given HIV C-peptides that inhibit HIV viral entry can also be used in the method set forth herein for enhancing anti-HIV potency. Variations in the amino acid residues of C-peptides that are envisioned for this invention are described in US Patent Application 2006/0247416 which is hereby incorporated by reference. The physical linking of a plurality of C-peptides produces an oligomeric C-peptide. In one embodiment, the C-peptide inhibitor is selected from the following group of C-peptides consisting of the amino acid sequences:

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;

(SEQ. ID. No. 5)

HTTWMEWDREINKYISLIHSLIEESQNQQEKNEQELL;
and

(SEQ. ID. No. 6)

WQEWEQKITALLEQAQIQQEKNEYELQKLDKWASLWEWF.

In one embodiment, the oligomeric HIV C-peptide inhibitors provided herein are assayed for their inhibition potency using methods also described herein. For example, the IC₅₀ for the oligomeric HIV C-peptide inhibitors against viral entry of the wild type and the resistant strains of HIV can be determined.

In one embodiment, a pharmaceutical composition comprising a composition of oligomeric peptides, peptide fragments and/or variants thereof that inhibit HIV viral entry, and an acceptable pharmaceutical carrier is provided. As used herein, the term “pharmaceutical composition” refers to the active agent in combination with a pharmaceutically acceptable carrier of chemicals and compounds commonly used in the pharmaceutical industry. The active agent used herein refers to the oligomeric C-peptides, peptide fragments and/or variants thereof that have inhibitory activity against HIV entry into host cells. The term “pharmaceutically acceptable carrier” excludes tissue culture medium. Such pharmaceutical compositions include solutions, suspensions, lotions, gels, creams, ointments, emulsions, skin patches, etc. All of these dosage forms, along with methods for their preparation, are known in the pharmaceutical and cosmetic art. Harry's Cosmeticology (Chemical Publishing, 7th ed. 1982); Remington's Pharmaceutical Sciences (Mack Publishing Co., 18th ed. 1990). Typically, topical formulations contain the active ingredient in a concentration range of 0.1 to 100 mg/ml, in admixture with suitable vehicles. A suitable pharmaceutically acceptable carrier will not promote an immune response to the oligomeric peptide constructs described herein. Other desirable ingredients for use in such preparations include preservatives, co-solvents, viscosity building agents, carriers, etc. The carrier itself or a component dissolved in the carrier may have palliative or therapeutic properties of its own, including moisturizing, cleansing, or anti-inflammatory/anti-itching properties. Penetration enhancers may, for example, be surface active agents; certain organic solvents, such as di-methylsulfoxide and other sulfoxides, dimethyl-acetamide and pyrrolidone; certain amides of heterocyclic amines, glycols (e.g. propylene glycol);propylene carbonate; oleic acid; alkyl amines and derivatives; various cationic, anionic, nonionic, and amphoteric surface active agents; and the like.

In one embodiment, an isolated nucleic acid construct is provided that encodes a protein comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined by a peptide linker. While it is possible to synthesize the oligomeric peptide by linking the individual isolated peptides. Recombinant methods known in the art can be use to synthesize the oligomeric peptide. The DNA coding sequence of a peptide can be amplified by PCR using the complete of the HR2 region of the HIV-1 transmembrane glycoprotein gp41 protein, amino acids 114-162 (SEQ. ID. No. 12) (Genbank Accession No. BD407105, NC_{—}001802, AJ293865) as a template for the PCR reaction. Specially designed PCR primers that incorporated restriction enzyme digestion sites and/or extra spacer or tag amino acid residues can be used to facilitate DNA ligation, recombinant protein expression, protein purification and protein identification. The amplified DNA coding sequence of a peptide can then be ligated to form a single coding DNA sequence that comprise several coding sequences of a peptide, ligated in tandem. Two and up to five coding sequences of a peptide are ligated in tandem. Additional amino acid coding sequences, coding for 2-10 amino acids, can be added between each pair of adjoining peptides as spacer sequences. When the single coding DNA sequence is transcribed and translated, the expressed polypeptide will contain tandem repeats of peptides, each separated by 2-10 extra amino acids. Typical amino acid residues used for spacing sequences are glycine, tyrosine, cysteine, lysine, proline, glutamic and aspartic acid, or the like. In a preferred embodiment, the oligomeric peptide is expressed copies of individual peptides arranged in an amino-carboxyl-amino-carboxyl tandem configuration. An example of an expressed oligomeric peptide comprising two copies of the same peptide with two glycine residues as spacer and a six histidine tag:

(SEQ. ID. No. 17)

HTTWMEWDREINNYTSLIHSLIEESQNQQEKNEQELLGGHTTWMEWDREI
NNYTSLIHSLIEESQNQQEKNEQELLGGHHHHHH.

In one embodiment, an isolated nucleic acid construct comprises the DNA coding sequences of peptides selected from the group consisting of:

(SEQ. ID. No. 2)

NYTSLIHSLIEESQNQQEKNEQELLELDKWASLWNWF;

(SEQ. ID. No. 3)

KYISLIHSLIEESQNQQEKNEQELLELDKWASLWNWF;

(SEQ. ID. No. 4)

HTTWMEWDREINNYTSLIHSLIEESQNQQEKNEQELL;

(SEQ. ID. No. 5)

HTTWMEWDREINKYISLIHSLIEESQNQQEKNEQELL;
and

(SEQ. ID. No. 6)

WQEWEQKITALLEQAQIQQEKNEYELQKLDKWASLWEWF.

In one embodiment, an isolated nucleic acid construct comprises two or more copies of a DNA coding sequence for a peptide. Accordingly, the encoded protein comprises repeated identical peptide sequences. For example, an isolated nucleic acid can comprise three tandemly ligated DNA sequences encoding NYTSLIHSLIEESQNQQEKNEQELLELDKWASLWNWF (SEQ. ID. No. 2) only. As a result, the expressed recombinant protein can be, for example, NYTSLIHSLIEESQNQQEKNEQELLELDKWASLWNWFxxxNYTSLIHSLIEESQNQQEKN EQELLELDKWASLWNWFxxxNYTSLIHSLIEESQNQQEKNEQELLELDKWASLWNWFxx xHHHHHH (SEQ. ID. No. 18) in a single polypeptide. The “xxx” represents spacer amino acid residues and the “HHHHHH” is the six-histidine tag useful for purification and identification of the expressed protein. In another embodiment, the isolated nucleic acid construct comprises the DNA coding sequence of different peptides. Accordingly, the encoded protein is comprised of different peptides. For example, the nucleic acid can comprise the DNA coding sequence for NYTSLIHSLIEESQNQQEKNEQELLELDKWASLWNWF (SEQ. ID. No. 2) and HTTWMEWDREINKYISLIHSLIEESQNQQEKNEQELL (SEQ. ID. No. 5) ligated tandemly. The expressed recombinant oligomeric protein can be, for example, NYTSLIHSLIEESQNQQEKNEQELLELDKWASLWNWFxxxHTTWMEWDREINKYISLIHS LIEESQNQQEKNEQELL (SEQ. ID. No. 19) in a single polypeptide.

In a preferred embodiment, the DNA coding sequence of the oligomeric peptide comprises two copies of DNA sequence encoding a peptide, three copies of DNA sequence encoding a peptide, four copies of DNA sequence encoding a peptide, or five copies of DNA sequence encoding a peptide. In a further preferred embodiment, the DNA coding sequence of the oligomeric peptide comprises two copies of DNA sequence encoding a peptide and/or three copies of DNA sequence encoding a peptide. Also envisioned is a DNA sequence encoding an oligomeric peptide comprising identical copies of a peptide, thus expressing a homo-oligomeric C-peptide inhibitor. Likewise, a DNA coding sequence for the oligomeric peptide can comprise copies of the DNA coding sequences of different and/or modified peptides, thus giving rise to a hetero-oligomeric C-peptide inhibitor. It is envisioned that all possible combination of DNA coding sequences for the different peptides can be ligated together to form a nucleic acid sequence that encodes an oligomeric C-peptide construct as described herein, e.g., oligomeric C-peptide constructs comprising HIV-inhibitory peptide HTTWMEWDREINNYTSLIHSLIEESQNQQEKNEQELLELDKWASLWNWF (SEQ. ID. No. 1) or fragments and/or variants thereof that inhibit HIV viral entry, wherein the plurality of peptides are physically joined amino acid residues.

In one embodiment, the invention disclosed herein provides a method for treating HIV infection that is caused by a strain that is resistant to an anti-HIV therapy in a subject, the method comprising administering to such subject, an effective amount of a composition with anti-HIV activity comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV entry, wherein the peptides are physically joined by a molecular linker.

In one embodiment, the methods and compositions disclosed herein are administered in conjunction with other anti-HIV therapies. The main strategy common to current HIV treatments is to combine different class of HIV inhibitors. One class of inhibitors are the HIV-specific protease inhibitors (PI) e.g. Ritonavir, Indivavir, tipranavir, and TMC114, and the other class of inhibitors is the highly active anti-retroviral therapy (HAART) that include reverse transcriptase inhibitors including nucleoside-analogue reverse transcriptase inhibitors (e.g, AZT, DDC, DDI and lamuvidine), and non-nucleoside-analogue reverse transcriptase inhibitors (e.g. Nevirapine). A more recent class of inhibitors is the HIV integrase inhibitors such as GS 9137 by discovered by Japan Tobacco, Inc (JT), MK-0518 and compounds described in U.S. Pat. No. 7,250,421 which is hereby incorporated by reference. The efficacy of the anti-HIV treatment is assessed by routine monitoring of the viral load and normal CD4+ T-cells counts in the blood stream.

When there is an increase in either the viral load and/or a decrease in normal CD4+ T-cell count in a subject being treated with a standard therapy, it can be an indication of the development of resistance to the anti-HIV therapy in use. Anti-retroviral drug resistance testing can performed to further verify the cause of resistance: (a) genotypic assays detect drug resistance mutations that are present in the relevant viral genes (i.e. RT and protease). Some genotyping assays involve sequencing of the entire RT and protease genes, while others utilize oligonucleotide probes to detect selected mutations that are known to confer drug resistance; and (b) phenotypic assays measure the ability of viruses to grow in the presence of various concentrations of antiretroviral drugs. Recombinant phenotyping assays involve insertion of the RT and protease gene sequences derived from patient plasma HIV RNA into a laboratory clone of HIV. Replication of the recombinant virus at various drug concentrations is monitored by expression of a reporter gene and is compared with replication of a reference strain of HIV. The concentrations of drugs that inhibit 50% and 90% of viral replication (i.e. the IC₅₀ and IC₉₀) are calculated, and the ratio of the IC₅₀s of the test and reference viruses is reported as the fold increase in IC₅₀, or fold resistance.

When drug resistances have been confirmed in a subject, that subject can be treated with a composition comprising an oligomeric peptide or an oligomeric C-peptide inhibitor as described herein. In a preferred embodiment, the composition is administered in combination with newer and more effective anti-HIV drugs of the PI class or the HAART reverse transcriptase inhibitors that have not been previously administered to the subject. In another preferred embodiment, the composition comprises a mixture of different oligomeric C-peptide inhibitors. In a further preferred embodiment, the mixture is of different hetero-oligomeric C-peptide inhibitors. The use of such a mixture of hetero-oligomeric C-peptide inhibitors together with another PI and/or HAART therapy serves to severely limit the opportunity for the virus to develop drug resistance to this combination of drug therapy.

In one embodiment, the invention provides a method of preventing the development of HIV resistance to anti-HIV therapy in a subject, the method comprising administering to such subject an effective amount of a composition with anti-HIV activity, the composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein the peptides are physically joined by a molecular linker, in combination with anti-HIV therapeutic of another class. Drug resistance develops as a result of the selection pressure imposed by the anti-HIV drugs. Each drug class targets the virus at a different process in its infectious life cycle. By using a combination of drugs from the different anti-HIV class, eg. PI, HAART, anti-fusion inhibition and anti-viral replication, a resistant strain emerging from the selection pressure of the PI or HAART drugs can be eliminated by the concomitant presence of the anti-fusion inhibitor and/or anti-viral replication. In one embodiment, the compositions described herein are administered with at least one anti-HIV drug from the PI, HAART class, or anti-viral replication class of anti-retroviral drugs. In a preferred embodiment, the compositions described herein are administered with two other anti-HIV drug, preferably from dissimilar classes. For example, treatment can be effected using a composition of oligomeric C-peptide inhibitors as described herein plus a new PI, or a composition of oligomeric C-peptides as described herein with two reverse transcriptase inhibitor nucleosides, as a model for drug-naive patients. In another preferred embodiment, the composition comprises a mixture of different oligomeric C-peptide inhibitors. In a further preferred embodiment, the mixture is of different hetero-oligomeric C-peptide inhibitors. In another embodiment, the anti-HIV drug can be from the group of HIV integrase inhibitors such as GS 9137.

Examples of the nucleoside reverse transcriptase inhibitors (NRTIs) are: abacavir, abacarvir sulfate, azidothymidine, dideoxycytidine, didexoyionsine, disoproxil, didanosine, emtricitabine, fumarate, lamivudine, tenofovir, stavudine, zalcitabine, and zidovudine. Examples of the Non nucleoside reverse transcriptase inhibitors (NNRTIs) are: delavirdine, efavirenz, and nevirapine. Examples of the protease inhibitors (PI) are amprenavir, atazanavir, darunavir, fosamprenavir calcium, indinavir, lopinavir, nelfinavir mesylate, ritonavir, saquinavir, saquainavir mesylate, and tipranavir. An example of fusion inhibitor is enfuvirtide.

As used herein, the development of resistance is “prevented” of an HIV viral infection fails to become resistant (i.e., if the efficacy of the anti-HIV therapy remains substantially constant) in an subject receiving the treatment with an oligomeric C-peptide inhibitor, or construct as described herein for the duration of the therapeutic intervention.

As used herein, the term “substantially” refers to less than 5% increase in HIV viral load and/or less than 5% decrease in CD4+ T-cell count compared to the previous viral load or T-cell monitoring conducted in a subject undergoing anti-HIV therapy. The subject has been on the anti-HIV therapy for a period of at least three months and the anti-HIV therapy has been effective in reducing viral load and increasing CD4+ T-cell counts in the subject. In a preferred embodiment, the increase in HIV viral load is less than 1% and the decrease in CD4+ T-cell count is less than 1%.

Synthesis of Peptides

Peptides described herein can be synthetically constructed by suitable known peptide polymerization techniques, such as exclusively solid phase techniques, partial solid-phase techniques, fragment condensation or classical solution couplings. For example, the peptides of the invention can be synthesized by the solid phase method using standard methods based on either t-butyloxycarbonyl (BOC) or 9-fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992) and in the textbook “Solid-Phase Synthesis”, Stewart & Young, Freemen & Company, San Francisco, 1969, and are exemplified by the disclosure of U.S. Pat. No. 4,105,603, issued Aug. 8, 1979. Classical solution synthesis is described in detail in “Methoden der Organischen Chemic (Houben-Weyl): Synthese von Peptiden”, E. Wunsch (editor) (1974) Georg Thieme Verlag, Stuttgart West Germany. The fragment condensation method of synthesis is exemplified in U.S. Pat. No. 3,972,859. Other available syntheses are exemplified in U.S., Pat. No. 3,842,067, U.S. Pat. No. 3,872,925, issued Jan. 28, 1975, Merrifield B, Protein Science (1996), 5: 1947-1951; The chemical synthesis of proteins; Mutter M, Int J Pept Protein Res 1979 March; 13 (3): 274-7 Studies on the coupling rates in liquid-phase peptide synthesis using competition experiments; and Solid Phase Peptide Synthesis in the series Methods in Enzymology (Fields, G. B. (1997) Solid-Phase Peptide Synthesis. Academic Press, San Diego.#9830). The foregoing disclosures are incorporated herein by reference.

In one embodiment, the HIV inhibitory C-peptides of the invention can be synthesized by recombinant molecular techniques that are known in the art. The expressed recombinant C-peptides and oligomeric C-peptides can contain natural amino acid residues.

Moreover, DNA coding sequence for the C-peptides, fragments, and/or variants thereof can be constructed by known techniques such as expression vectors or plasmids and transfected into suitable microorganisms that will express the DNA sequences thus preparing the peptide for later extraction from the medium in which the microorganisms are grown. For example, U.S. Pat. No. 5,595,887 describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

Recombinant techniques are well known to those skilled in the art. Representative methods are disclosed in Maniatis, et al., Molecular cloning, a Laboratory Manual, 2nd edition, Cold Springs Harbor Laboratory (1989), incorporated herein by reference. As mentioned above, recombinant DNA synthesis can be used to produce not only the individual peptide, but also an oligomeric C-peptide comprising several C-peptides.

The DNA coding sequence of a peptide can be amplified by PCR using the complete HR2 region of the HIV-1 transmembrane glycoprotein gp41 protein, amino acids 114-162 (SEQ. ID. No.) (Genbank Accession No. BD407105, NC_{—}001802, AJ293865) as a template for the PCR reaction. Specially designed PCR primers that incorporate restriction enzyme digestion sites and/or extra spacer or tag amino acid residues can be used to facilitate DNA ligation, recombinant protein expression, protein purification and protein identification. The amplified DNA coding sequence of a peptide can then be ligated to form a single coding DNA sequence that comprises a plurality of coding sequences of the peptide, ligated in tandem. Two and up to five coding sequences of a peptide are ligated in tandem. Additional amino acid coding sequences, coding for 2-10 amino acids, can be added between each pair of adjoining peptides as spacer sequences. When the single coding DNA sequence is transcribed and translated, the expressed polypeptide will contain tandem repeats of peptides, each separated by 2-10 extra amino acids. Typical amino acid residues used for spacing sequences are glycine, tyrosine, cysteine, lysine, proline, glutamic and aspartic acid, or the like. In a preferred embodiment, the oligomeric peptide is expressed as copies of individual peptides arranged in an amino-carboxyl-amino-carboxyl tandem configuration.

Conventional polymerase chain reaction (PCR) cloning techniques can be used to generate an isolated DNA sequence encoding a peptide. The polymerase used in the PCR amplification should have high fidelity such as Strategene's PfuUltra™ polymerase for reducing sequence mistakes during the PCR amplification process. Restriction digestion sites can be incorporated into the PCR primers and preferably different restriction digestion sites are used for the 5′ PCR primer and the 3′ PCR primer in order to facilitate asymmetrical ligation into a cloning or expression vector. A general purpose cloning vector such as pUC19, pBR322, pBluescript vectors (Stratagene Inc.) or pCR TOPO® from Invitrogen Inc. can be used for cloning.

Alternatively the isolated DNA sequence encoding a C-peptide can be ligated into a vector using the TOPO® cloning method in Invitrogen topoisomerase-assisted TA vectors such as pCR®-TOPO, pCR®-Blunt II-TOPO, pENTR/D-TOPO®, and pENTR/SD/D-TOPO®. Both pENTR/D-TOPO®, and pENTR/SD/D-TOPO® are directional TOPO entry vectors which allow the cloning of the C-peptide DNA coding sequence in the 5′→3′ orientation into a Gateway® expression vector. Directional cloning in the 5′→3′ orientation facilitates the unidirectional insertion of the C-peptide DNA sequence into a protein expression vector such that the promoter is upstream of the 5′ ATG start codon of the C-peptide DNA coding sequence, enabling promoter driven protein expression. The recombinant vector carrying the C-peptide DNA coding sequence can be transfected into and propagated in general cloning E. coli such as XL1Blue, SURE (Stratagene) and TOP-10 cells (Invitrogen).

The resultant recombinant vector carrying the C-peptide DNA coding sequence can then be used for further molecular biological manipulations such as site-directed mutagenesis to create specific amino acid mutations and substitutions in the C-peptide, thus producing variant forms of C-peptides, or can be subcloned into protein expression vectors or viral vectors for protein synthesis in a variety of protein expression systems using host cells selected from the group consisting of mammalian cell lines, insect cell lines, yeast, bacteria, and plant cells. Examples of amino acid mutations are serine to proline mutation and a substitution of leucine for glutamate. Site-directed mutagenesis may be carried out using the QuikChange® site-directed mutagenesis kit from Stratagene according to the manufacturer's instructions or any methods known in the art.

In a preferred embodiment, the vector is an expression vector adapted for prokaryotic or eukaryotic cell expression. Adaptations to the expression vector can be envisioned to facilitate efficient protein expression of the recombinant protein. Typically adaptation includes, by example and not by way of limitation, the provision of transcription control sequences (promoter sequences) which mediate cell/tissue specific expression. These promoter sequences may be cell/tissue specific, inducible or constitutive.

Promoter is an art recognized term and, for the sake of clarity, includes the following features which are provided by example only, and not by way of limitation. Enhancer elements are cis-acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S. Latchman, Academic Press Ltd, San Diego) is responsive to a number of environmental cues which include, by example and not by way of limitation, intermediary metabolites or environmental effectors. Promoter elements also include so called TATA box and RNA polymerase initiation selection (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) which function to maximize expression of vector encoded genes arranged in bicistronic or multi-cistronic expression cassettes.

Expression Vectors and Expression Systems

In one embodiment, the invention provides for expression vectors carrying a DNA coding sequence that encodes a C-peptide or an oligomeric C-peptide for the expression and purification of the recombinant C-peptide or oligomeric C-peptide produced from a protein expression system using host cells selected from, e.g., mammalian, insect, yeast, bacterial, or plant cells.

In one embodiment, the recombinant vector that expresses the C-peptide or oligomeric C-peptide is a viral vector. The viral vector can be any viral vector known in the art including but not limited to those derived from adenovirus, adeno-associated virus (AAV), retrovirus, and lentivirus. Recombinant viruses provide a versatile system for gene expression studies and therapeutic applications.

In another embodiment, the invention provides for a host cell comprising an expression vector which expresses a C-peptide or an oligomeric C-peptide. The expression host cell may be derived from any of a number of sources, e.g., bacteria, such as E. coli, yeasts, mammals, insects, and plant cells such as Chlamydomonas. In another embodiment, the recombinant C-peptide or an oligomeric C-peptide can be produced from expression vectors suitable for cell-free expression systems. From the cloning vector, the C-peptide DNA coding sequence can be subcloned into a recombinant expression vector that is appropriate for the expression of the C-peptide or an oligomeric C-peptide in mammalian, insect, yeast, bacterial, or plant cells or a cell-free expression system such as a rabbit reticulocyte expression system. Subcloning can be achieved by PCR cloning, restriction digestion followed by ligation, or recombination reaction such as those of the lambda phage-based site-specific recombination using the Gateway® LR and BP Clonase™ enzyme mixtures. Subcloning should be unidirectional such that the 5′ ATG start codon of the C-peptide DNA sequence is downstream of the promoter in the expression vector. Alternatively, when the C-peptide DNA sequence is cloned into pENTR/D-TOPO®, pENTR/SD/D-TOPO® (directional entry vectors), or any of the Invitrogen's Gateway® Technology pENTR (entry) vectors, the C-peptide DNA sequence can be transferred into the various Gateway® expression vectors (destination) for protein expression in mammalian cells, E. coli, insects and yeast respectively in one single recombination reaction. Some of the Gateway® destination vectors are designed for the constructions of baculovirus, adenovirus, adeno-associated virus (AAV), retrovirus, and lentiviruses, which upon infecting their respective host cells, permit heterologous expression of the C-peptide-binding protein in the host cells. The Gateway® Technology uses lambda phage-based site-specific recombination instead of restriction endonuclease and ligase to insert a gene of interest into an expression vector. The DNA recombination sequences (attL, attR, attB, and attP) and the LR and BP Clonase™ enzyme mixtures that mediate the lambda recombination reactions are the foundation of Gateway® Technology. Transferring a gene into a destination vector is accomplished in just two steps: Step 1: Clone the chimeric DNA sequence into an entry vector such as pENTR/D-TOPO®. Step 2: Mix the entry clone containing the chimeric DNA sequence in vitro with the appropriate Gateway® expression vector (destination vector) and Gateway® LR Clonase™ enzyme mix. There are Gateway® expression vectors for protein expression in E. coli, insect cells, mammalian cells, and yeast. Site-specific recombination between the att sites (attR x attL and attB x attP) generates an expression vector and a by-product. The expression vector contains the C-peptide DNA coding sequence recombined into the destination vector backbone. Following transformation and selection in E. coli, the expression vector is ready to be used for expression in the appropriate host.

The expression vector should have the necessary 5′ upstream and 3′ downstream regulatory elements such as promoter sequences, ribosome recognition and binding TATA box, and 3′ UTR AAUAAA transcription termination sequence for the efficient gene transcription and translation in its respective host cell. The expression vector may have additional sequence such as 6×-histidine, V5, thioredoxin, glutathione-S-transferase, c-Myc, VSV-G, HSV, FLAG, maltose binding peptide, metal-binding peptide, HA and “secretion” signals (Honeybee melittin, α-factor, PHO, Bip), which are incorporated into the expressed recombinant C-peptide or an oligomeric C-peptide. In addition, there may be enzyme digestion sites incorporated after these sequences to facilitate enzymatic removal of them after they are not needed. These additional sequences are useful for the detection of the C-peptide or an oligomeric C-peptide expression, for protein purification by affinity chromatography, enhanced solubility of the recombinant protein in the host cytoplasm, and/or for secreting the expressed recombinant C-peptide or an oligomeric C-peptide out into the culture media, into the periplasm of the prokaryote bacteria, or the spheroplast of the yeast cells. The expression of the recombinant C-peptide or an oligomeric C-peptide can be constitutive in the host cells or it can be induced, e.g., with copper sulfate, sugars such as galactose, methanol, methylamine, thiamine, tetracycline, infection with baculovirus, and (isopropyl-beta-D-thiogalactopyranoside) IPTG, a stable synthetic analog of lactose.

Examples of expression vectors and host cells are the pET vectors (Novagen), pGEX vectors (Amersham Pharmacia), and pMAL vectors (New England labs. Inc.) for protein expression in E. coli host cell such as BL21, BL21(DE3) and AD494(DE3)pLysS, Rosetta (DE3), and Origami(DE3) (Novagen); the strong CMV promoter-based pcDNA3.1 (Invitrogen) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells; BACpak6 baculovirus (Clontech) and pFastBac™ HT (Invitrogen) for the expression in Spodopera frugiperda 9 (Sf9) and Sf11 insect cell lines; pMT/BiP/V5-His (Invitrogen) for the expression in Drosophila Schneider S2 cells; Pichia expression vectors pPICZα, pPICZ, pFLDα and pFLD (Invitrogen) for expression in Pichia pastoris and vectors pMETα and pMET for expression in P. methanolica; pYES2/GS and pYD1 (Invitrogen) vectors for expression in yeast Saccharomyces cerevisiae. Recent advances in the large scale expression heterologous proteins in Chlamydomonas reinhardtii are described by Griesbeck C. et. al. 2006 Mol. Biotechnol. 34:213-33 and Fuhrmann M. 2004, Methods Mol. Med. 94:191-5. Foreign heterologous coding sequences are inserted into the genome of the nucleus, chloroplast and mitochodria by homologous recombination. The chloroplast expression vector p64 carrying the most versatile chloroplast selectable marker aminoglycoside adenyl transferase (aadA), which confer resistance to spectinomycin or streptomycin, can be used to express foreign protein in the chloroplast. Biolistic gene gun method is used to introduced the vector in the algae. Upon its entry into chloroplasts, the foreign DNA is released from the gene gun particles and integrates into the chloroplast genome through homologous recombination.

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, the invention provides a recombinant lentivirus for the delivery and expression of a C-peptide-binding protein in either dividing and non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with ViraPower™ Lentiviral Expression systems from Invitrogen.

In one embodiment, the invention provides a recombinant adeno-associated virus (rAAV) vector for the expression of a C-peptide or an oligomeric C-peptide. In one embodiment, the rAAV vector encoding a C-peptide or an oligomeric C-peptide can be used to infect huma cell lines from the large scale production of recombinant proteins. In another embodiment, the vector can be administered to a subject infected with HIV. Using rAAV vectors, genes can be delivered into a wide range of host cells including many different human and non-human cell lines or tissues. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 1011-1012 viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the C-peptide DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients.

Expression and Purification

In one embodiment, the invention provides a method of producing C-peptide or oligomeric C-peptide comprising introducing the recombinant vector that expressed the C-peptide or oligomeric C-peptide into an isolated host cell, growing the cell under conditions permitting the production of the recombinant protein and recovering the recombinant protein so produced. The methods described herein provide for the expression and purification of the C-peptide or oligomeric C-peptide in various cell-based expression systems such as protein production in bacterial, mammalian, insect, yeast, and chymadomonas cells are well known in the art. Protein expression can be constitutive or inducible with inducers such as copper sulfate, sugars such as galactose, methanol, methylamine, thiamine, tetracycline, or IPTG. After the protein is expressed in the host cells, the host cells are lysed to liberate the expressed protein for purification. Methods of lysing the various host cells are featured in “Sample Preparation-Tools for Protein Research” EMD Bioscience and in the Current Protocols in Protein Sciences (CPPS). The preferred purification method is affinity chromatography such as ion-metal affinity chromatograph using nickel, cobalt, or zinc affinity resins for histidine-tagged C-peptide-binding protein. Methods of purifying histidine-tagged recombinant proteins are described by Clontech using their Talon® cobalt resin and by Novagen in their pET system manual, 10th edition. Another preferred purification strategy is by immuno-affinity chromatography, for example, anti-myc antibody conjugated resin can be used to affinity purify myc-tagged C-peptide or oligomeric C-peptide. Enzymatic digestion with serine proteases such as thrombin and enterokinase cleave and release the C-peptide-binding protein from the histidine or myc tag, releasing the C-peptide-binding protein from the affinity resin while the histidine-tags and myc-tags are left attached to the affinity resin.

Besides cell-based expression systems, cell-free expression systems are also contemplated. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix.

In one embodiment, a continuous cell-free translation system may be used to produce a C-peptide or oligomeric C-peptide. A continuous cell-free translation system capable of producing proteins in high yield is described by Spirin A S. et. al., Science 242:1162 (1988). The method uses a continuous flow design of the feeding buffer which contains amino acids, adenosine triphosphate (ATP), and guanosine triphosphate (GTP) throughout the reaction mixture and a continuous removal of the translated polypeptide product. The system uses E. coli lysate to provide the cell-free continuous feeding buffer. This continuous flow system is compatible with both prokaryotic and eukaryotic expression vectors. Large scale cell-free production of the integral membrane protein EmrE multidrug transporter is described by Chang G. el. al., Science 310:1950-3 (2005).

Other commercially available cell-free expression systems include the Expressway™ Cell-Free Expression Systems (Invitrogen) which utilize an E. coli-based in-vitro system for efficient, coupled transcription and translation reactions to produce up to milligram quantities of active recombinant protein in a tube reaction format; the Rapid Translation System (RTS) (Roche Applied Science) which also uses an E. coli-based in-vitro system; and the TNT Coupled Reticulocyte Lysate Systems (Promega) which uses rabbit reticulocyte-based in-vitro system.

Chemical Cross-Linking to Form Oligomeric Peptides

The physical linking of the individual isolated peptides into oligomeric peptides as set forth herein, can be effected by chemical conjugation procedures well known in the art, such as by creating peptide linkages, use of condensation agents, and by employing well known bifunctional cross-linking reagents. The conjugation may be direct, which includes linkages not involving any intervening group, e.g., direct peptide linkages, or indirect, wherein the linkage contains an intervening moiety, such as a protein or peptide, e.g., plasma albumin, or other spacer molecule. For example, the linkage may be via a heterobifunctional or homobifunctional cross-linker, e.g., carbodiimide, glutaraldehyde, N-succinimidyl 3-(2-pyridydithio) propionate (SPDP) and derivatives, bis-maleimide, 4-(N-maleimidomethyl)cyclohexane-1-carboxylate, and the like.

Cross-linking may also be accomplished without exogenous cross-linkers by utilizing reactive groups on the molecules being conjugated. Methods for chemically cross-linking peptide molecules are generally known in the art, and a number of hetero- and homobifunctional agents are described in, e.g., U.S. Pat. Nos. 4,355,023, 4,657,853, 4,676,980, 4,925,921, and 4,970,156, and Immuno Technology Catalogue and Handbook, Pierce Chemical Co. (1989), each of which is incorporated herein by reference. Such conjugation, including cross-linking, should be performed so as not to substantially affect the desired function of the peptide oligomer or entity conjugated thereto, including therapeutic agents, and moieties capable of binding substances of interest.

Conjugation of individual peptide can be effected by a linkage via the N-terminal or the C-terminal of the peptide, resulting in an N-linked peptide oligomer or a C-linked peptide oligomer, respectively.

It will be apparent to one skilled in the art that alternative linkers can be used to link peptides, for example the use of chemical protein crosslinkers. For example homobifunctional crosslinker such as disuccinimidyl-suberimidate-dihydrochloride; dimethyl-adipimidate-dihydrochloride; 1,5,-2,4-dinitrobenezene or heterobifunctional crosslinkers such as N-hydroxysuccinimidyl 2,3-dibromopropionate; lethyl-3-[3-dimethylaminopropyl]carbodiimide hydrochloride; and succinimidyl-4-[n-maleimidomethyl]-cyclohexane-1-carboxylate.

Formulation and Composition

In one embodiment, the invention described herein comprises a pharmaceutical composition comprising a composition of oligomeric peptides and/or oligomeric C-peptide inhibitors and a pharmaceutically acceptable carrier.

Dosage forms of the pharmaceutical composition, along with methods for their preparation, are well known in the pharmaceutical and cosmetic art (see HARRY'S COSMETICOLOGY (Chemical Publishing, 7th ed. 1982); REMINGTON'S PHARMACEUTICAL SCIENCES (Mack Publishing Co., 18th ed. 1990)). Other desirable ingredients for use in such preparations include preservatives, co-solvents, viscosity building agents, carriers, etc. The carrier itself or a component dissolved in the carrier may have palliative or therapeutic properties of its own, including moisturizing, cleansing, or anti-inflammatory/anti-itching properties. Penetration enhancers may, for example, be surface active agents; certain organic solvents, such as di-methylsulfoxide and other sulfoxides, dimethyl-acetamide and pyrrolidone; certain amides of heterocyclic amines, glycols (e.g. propylene glycol); propylene carbonate; oleic acid; alkyl amines and derivatives; various cationic, anionic, nonionic, and amphoteric surface active agents; and the like.

In one embodiment, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. Nos. 3,773,919, 3,887,699, EP 58,481A, EP 158,277A, Canadian Patent No. 1176565, U. Sidman et al., Biopolymers 22:547 (1983) and R. Langer et al., Chem. Tech. 12:98 (1982). Various controlled release systems, such as monolithic or reservoir-type microcapsules, depot implants, osmotic pumps, vesicles, micelles, liposomes, transdermal patches, iontophoretic devices and alternative injectable dosage forms may be used for this purpose.

Proteins will usually be formulated at a concentration of about 0.1 mg/ml to 100 mg/ml. Viral vectors that carry the gene for expressing biologics in vivo should be in the range of 10⁶ to 1×10¹⁴ viral vector particles per application per patient.

In one embodiment, other ingredients can be added to pharmaceutical formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the pharmaceutical formulation to be used for therapeutic administration must be sterile. Sterility is readily accomplished by filtration through sterile filtration membranes (e.g., 0.2 micron membranes). The oligomeric peptides ordinarily can be stored in lyophilized form or as an aqueous solution if it is highly stable to thermal and oxidative denaturation. The pH of the oligomeric peptide preparations typically will be about from 6 to 8, although higher or lower pH values may also be appropriate in certain instances.

The pharmaceutical compositions described herein can also be administered systemically in a pharmaceutical formulation. The preferred formulation is also sterile saline or Lactated Ringer's solution. Lactated Ringer's solution is a solution that is isotonic with blood and intended for intravenous administration. Systemic routes include but are not limited to oral, parenteral, nasal inhalation, intratracheal, intrathecal, intracranial, and intrarectal. The pharmaceutical formulation is preferably a sterile saline or lactated Ringer's solution. For therapeutic applications, the preparations described herein are administered to a mammal, preferably a human, in a pharmaceutically acceptable dosage form, including those that may be administered to a human intervenously as a bolus or by continuous infusion over a period of time, by intramuscular, intraperitoneal, intracerebrospinal, subcutaneous, intra-arterial, intrasynovial, intrathecal, oral, topical, or inhalation routes. The oligomeric peptide can be formulated for nasal inhalation using a nebulizer. Viral vectors encoding an oligomeric peptide can be formulated for use with a nebulizer. For these uses, additional conventional pharmaceutical preparations such as tablets, enteric coated tablets or capsules, granules, powders, capsules, and sprays may be preferentially required. In such formulations further conventional additives such as binding-agents, wetting agents, propellants, lubricants, and stabilizers may also be required. In one embodiment, the therapeutic compositions described herein are formulated in a cationic liposome formulation such as those described for intratracheal gene therapy treatment of early lung cancer (Zou Y. et. al., Cancer Gene Ther. 2000 May; 7(5):683-96). The liposome formulations are especially suitable for aerosol use for delivery to the lungs of patients. Vector DNA and/or virus can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et. al. Gene Ther. 1999, 6:1438-47). Other techniques in formulating expression vectors and virus as therapeutics are found in “DNA-Pharmaceuticals: Formulation and Delivery in Gene Therapy, DNA Vaccination and Immunotherapy” by Martin Schleef (Editor) December 2005, Wiley Publisher, and “Plasmids for Therapy and Vaccination” by Martin Schleef (Editor) May 2001, are incorporated herein as reference. In one embodiment, the dosage for viral vectors is 10⁶ to 10¹⁴ viral vector particles per application per patient.

The route of administration, dosage form, and the effective amount vary according to the potency of the oligomeric peptide, and expression vectors and viral vectors used the gene therapy, and their physicochemical characteristics. The selection of proper dosage is well within the skill of an ordinarily skilled physician.

This invention is further illustrated by the following example which should not be construed as limiting. The contents of all references cited throughout this application, as well as the figures and table are incorporated herein by reference.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

### Example

### Introduction

C-peptides inhibit gp41 in a kinetic window between CD4-gp120 interaction and trimer-of-hairpins formation. As a consequence, their potency is not only dependent on binding affinity, but is also influenced by kinetic parameters such as the rate of association of C-peptides with gp41 and the lifetime of the sensitive intermediate state. These kinetic parameters tend to limit T20 and C37 potency to the low nanomolar range (variable depending on viral strain and infectable target cells). Tighter binding variants of T20 and C37 tend to inhibit wild type virus with the same nanomolar potencies.

Resistance to C-peptides develops through at least two different mechanisms. The first is straightforward and much more commonly observed: resistant viruses tend to accumulate mutations in the gp41 HR1 region, especially in the sequence between amino acids 543 and 552-QLLSGIVQQQ (SEQ. ID. No. 13) in HXB2 sequence, that substantially reduce T20 and C37 binding affinity. Two commonly observed resistant profiles are QLLSDTVQQQ (SEQ. ID. No. 14)and QLLSGIEQQQ (SEQ. ID. No. 15), where the negative charge of the introduced Asp or Glu appears to substantially disrupt T20 and C37 binding. The second resistance profile, observed less frequently, involves mutations in the HR2 region of gp41. The mechanism behind this profile has not been fully ascertained, but research work on a different gp41 inhibitor (5-Helix) indicate that a subset of mutations lead to the decrease in the lifetime of gp41 intermediate states, thereby reducing the amount of time C-peptides have to bind to gp41. This subset of mutations involves conserved glycosylation sites formed by Asn637 and Thr639.

Experimental Procedures

C-Peptide Syntheses

The recombinant C37 utilized in the study contain HR2 residues 625-661 from either HXB2 or JRFL Env (numbering according to the HXB2 sequence). Expression vectors for C37 recombinant polypeptide variants were generated by QuickChange site-directed mutagenesis (Stratagene) and confirmed by sequencing the entire open reading frame. C-peptide C37 and its variants (N637K, T6391, N637K/T6391 and T639S) were obtained by cleavage of a recombinantly-expressed trimer-of-hairpins construct (CGG-NC1.1), followed by reverse-phase high-pressure liquid chromatography (rpHPLC), also described previously (Root, M., et. al., (2001) Science 291, 884-888). T20 was synthesized using standard Fmoc chemistry in the Proteomics/Peptide Synthesis Facility of the Kimmel Cancer Center (Thomas Jefferson University, Philadelphia). Desalted peptide was purified to homogeneity by rpHPLC using a Vydac C-18 column and a linear gradient of acetonitrile in water containing 0.1% trifluoroacetic acid. The identities of purified T20 and C37 peptides were confirmed using SELDI-TOF mass spectrometry (Ciphergen). Concentrations for all polypeptides were determined by absorbance at 280 nm in 6 M guanidine HCl (GuHCl) (Edelhoch, H., 1967, Biochemistry 6, 1948-1954).

C37 peptide was diluted to 4 mg of protein per ml in 100 mM potassium phosphate, pH 7.2/10 mM MgSO₄ and divided into 0.4-ml aliquots. After addition of MTS-3-MTS (5 Å) or 3,6,9,12,15-pentaoxaheptadecane-1,17-diyl bis-methanethiosulfonate (MTS-17-05-MTS; 21 Å) in DMSO to a final concentration of 50 μM, the samples were incubated for 1 h at room temperature. The same volume of DMSO was added to the control sample (final concentration 0.25%). The cross-linking reaction was terminated by addition of methylmethane thiosulfonate to a final concentration of 5 mM. After harvesting by centrifugation for 2 min at 10,000×g, the vesicles were washed twice with 100 mM potassium phosphate (pH 7.2)/10 mM MgSO₄ and resuspended in 0.4 ml of the same buffer. An aliquot (40 μl) of each sample was subjected to SDS/glycerol/18% PAGE.

Inhibition Assays and Viral Infection Assay.

All viral infectivity and cell-cell fusion experiments were performed as described in Chan, D., et. al., 1998, Proc. Natl. Acad. Sci. U.S.A. 95, 15613-15617. Briefly, the potency of C-peptides in inhibiting viral infection was determined using recombinant luciferase-expressing HIV-1 as described by Malashkevich, V. N., et. al., 1998, Proc. Natl. Acad. Sci. USA 95, 9134-9139. To produce virus, 293T cells were co-transfected with the envelope-deficient HIV-1 genome NL43LucR-E-(21) and the HXB2 gp160 expression vector pCMVHXB2 gp160 using calcium phosphate. Viral supernatants were cleared of cellular debris by low-speed centrifugation and used to infect HOS-CD4/Fusin cells (N. Landau, National Institutes of Health AIDS Reagent Program) in the presence of varying concentrations of C-peptide, ranging from 0 to 200 nM. Cells were harvested 48 hr postinfection, and luciferase activity was measured in a Wallac (Gaithersburg, Md.) AutoLumat LB953 luminometer. The IC₅₀ is the peptide concentration that results in a 50% decrease in activity relative to control samples lacking peptide. For each peptide, data from three experiments were fit to a Langmuir equation [y=k/(1+([peptide]/IC₅₀)], where y=luciferase activity and k is a scaling constant] to obtain the IC₅₀ values.

Syncytia formation was assayed by coculturing the HXB2 envelope-expressing cell line Chinese hamster ovary [HIVe](clone 7d2) (22) with the CD4-expressing cell line HeLa-CD4-LTR-Beta-gal (M. Emerman, National Institutes of Health AIDS Reagent Program) in the presence of varying concentrations of peptide, ranging from 0 to 200 nM. Cell fusion results in expression of nuclear-galactosidase from the HeLa-CD4-LTR-Beta-gal indicator cell line. Fifteen hours after coculture, monolayers were stained with the colorimetric substrate 5-bromo-4-chloro-3-indolyl—D-galactoside, and syncytia formation was quantitated by counting multinucleated cells containing at least three-galactosidase-positive nuclei. For each peptide, data from three experiments were fit to a Langmuir equation to obtain the IC₅₀ values.

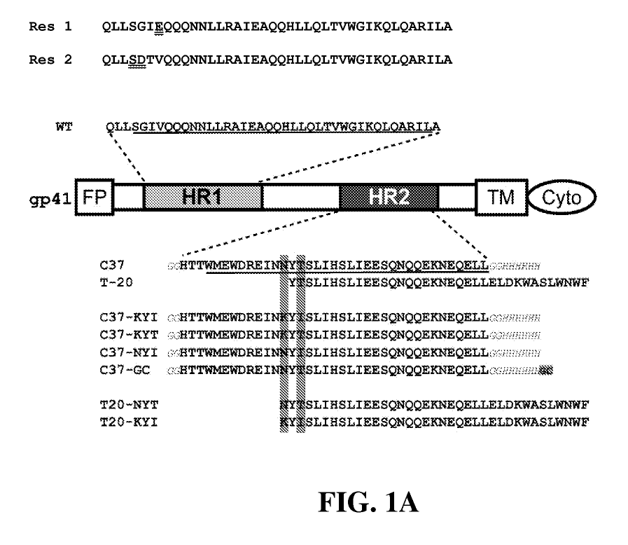

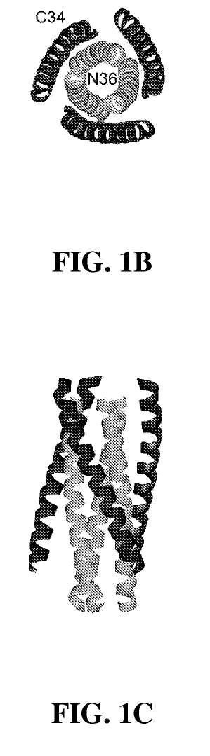

HOS-CD4-Les cells were infected with HIV-1 pseudotyped with Env from HXB2 HIV-1 strain of either the wild type sequence or one of three variant sequences that confer resistance to C-peptides. The sequences of Res1 and Res2 Env, defined in FIG. 1, contain mutations in the gp41 HR1 region that decrease C-peptide binding affinity. The sequence of Res3 contains a mutation in the gp41 HR2 region (N637K) that confers partial resistance by enhancing the rate of viral membrane fusion. The IC₅₀ (±SEM) values represent the mean of 2 or 3 independent titrations of the indicated C-peptide.

Results

FIG. 1A shows the schematic diagram of gp41 showing the HR1 and HR2 regions in the context of the fusion peptide (FP), transmembrane region (TM) and cytoplasmic tail (Cyto). Sequences for the HR1 (WT) and HR2 (C37 and T20) are shown in bold above and below the schematic, respectively. The smaller, italicized residues in C37 sequence are not found in gp41 but are included in the inhibitory peptide. Res1 and Res2 sequences above the HR1 sequence are derived from the two C37- and T20-resistant strains used in this study, where the double underlined amino acid residues identify the escape mutations. The sequences of the indicated C37 and T20 variants are shown, the residues Asn637 to lys637 (N→K) mutation and Thr639 to isoleucine (T→I) mutation are boxed and the added C-terminus Gly-Cys are underlined in bold.

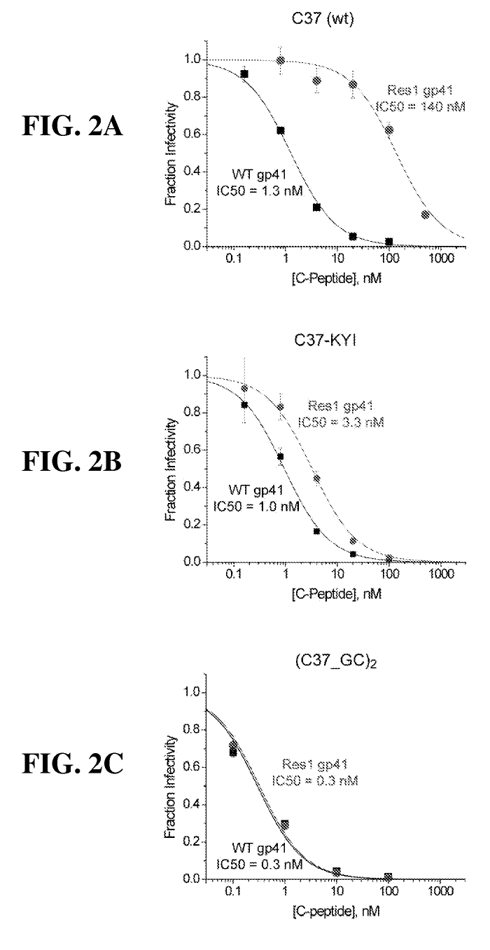

The C37 variants generated were able to overcome resistance conferred by the HR1 region mutations. In FIG. 2, viruses pseudotyped with either wild type Env (closed squares) or V549E mutant Env (Res1, closed circles) from strain HXB2 were utilized to infect HOS-CD4-Les target cells. Infection took place in the presence and absence of C37 (FIG. 2A), C37-KYI (FIG. 2B) or (C37-GC)₂ (FIG. 2C) and quantified by the expression of a luciferase-reporter construct. The IC₅₀ values are indicated on the graph. A mutant variant of C37 with two substitutions (N637K and T6391), hereafter denoted C37-KYI, binds to a structural mimic of the gp41 HR1 region, the N-peptide known as N36 with an estimated equilibrium dissociation constant (K_{d}) of 40 fM, more than 20-fold lower than the K_{d} for wild type C37 (FIG. 2). The Hr1 and HR2 regions of three gp41 form an intermediate structure called the trimer-of-hairpins which is necessary for the viral entry process and membrane fusion with the host membrane. FIGS. 1B and 1C showed the spatial relation between the HR1 and the HR2 of gp41, and the mimic peptides of HR1 and HR2, N36 and C37 peptides.

While C37-KYI inhibits viruses psuedotyped with EnvHXB2 with the same potency (IC₅₀˜1 nM) as C37, the potency of the variant peptide is only slightly diminished in the setting of the two HR1 region substitutions listed above. By contrast, the IC₅₀ values for the original C37 peptide are increased 40- to 110-fold. A T20 variant with the same KYI substitution showed a similar increase in binding affinity.

The disulfide-crosslinked dimeric C37 variant containing a Cys residue at the peptide's C-terminus (C37-GC) was able to overcome resistance conferred by the HR1 region mutations (FIG. 2C). Consistent with the kinetic restriction of inhibition data, the cross-linked dimeric C37-GC peptide was not significantly better at inhibiting wild type virus compared to the monomeric C37 peptide (Table 1). However, the IC₅₀ for the cross-linked peptide was unaffected by the resistance mutations at the HR1 region that confer a 100-fold reduction in monomeric C37 inhibitory potency (Table 1). This enhanced activity of the cross-linked C37 against resistant viruses was attributed to an avidity effect: although each C37 peptide alone bound more weakly to the HR1 region with escape mutations, the binding of the linked pair overcame this loss of affinity. Similarly, the cross-linked T20 showed more than 20 fold enhanced inhibition activity against the resistant strain Res4 (having the SDTV mutation) compared to the monomeric T20 peptide against the same resistant HIV (Table 2).

Resistance to antiviral agents is a significant problem in the treatment of HIV-1 infection. The modifications detailed above point to a general strategy to overcome common resistance profiles for C-peptide inhibitors of gp41: increase inhibitor binding strength through enhanced affinity or avidity. The field has been searching for “better” C-peptides, but their benchmarks to date have been mostly focused on improving pharmacokinetic properties and enhancing inhibitor potency against wild type virus. Because C-peptides most likely interact with wild type gp41 as fast as theoretically possible, the kinetic restriction on inhibition has prevented finding C-peptides with lower IC₅₀ for wild type virus. Thus, the higher affinity C-peptide variants developed here inhibit the wild type virus as potently as the original C37 and T20 do. However, they are substantially better at inhibiting virus resistant to the original peptides.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 1
Inhibitory potencies of C-peptides against
wild type and resistant HIV-1 strains

| | ENV (gp120/gp41) Glycoproteins | | | |
|---|---|---|---|---|
| | | HR1 Mutants | | HR2 Mutant |
| C-peptides | WT | Res 1 | Res 2 | Res 3 |
| C37 | 1.3 ± 0.2 | 126 ± 24 | 53 ± 15 | 7.1 ± 3.6 |
| T20 | 1.7 ± 0.4 | | 900 ± 150 | 6.4 ± 1.5 |
| C37-KYI | 1 ± 0.1 | 4.2 ± 1 | 0.6 ± 0.1 | 4.1 ± 1 |
| (C37-GC)₂ | 0.3 | 0.3 | | |

TABLE 2
Inhibitory potencies of C-peptides against
wild type and resistant HIV-1 strains

| Virus | C37 WT, | C37 KYI, | T20 WT, | polyT20, |
|---|---|---|---|---|
| Genotype | IC₅₀, nM | IC₅₀, nM | IC₅₀, nM | IC₅₀, nM |
| WT HXB2 | 1.3 ± 0.2 | 1.0 ± 0.1 | 2.3 ± 1.0 | 143 ± 41 |
| Res 1 (V38E) | 126 ± 24 | 4.2 ± 1.0 | ND | ND |
| Res 2 (L33S) | 1.3 ± 0.4 | 1.0 ± 0.2 | ND | ND |
| Res 3 | 7.1 ± 3.6 | 4.1 ± 1.0 | ND | ND |
| (N123K/L204I) | | | | |
| Res 4 (SDTV) | 53 ± 15 | 0.6 ± 0.1 | 900 ± 150 | 38 ± 17 |

ND = not done

1. A composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein said plurality of peptides are physically joined by a molecular linker.

2. The composition of claim 1, wherein the composition is a dimer of two said peptides.

3. The composition of claim 1, wherein the composition is a trimer of three said peptides.

4. The composition of claim 1, wherein the peptides are identical.

5. The composition of claim 1, wherein the peptides are different.

6. The composition of claim 1, wherein the molecular linker is a peptide linker molecule.

7. The composition of claim 6, wherein the peptide linker comprises at least 2 amino acids residues.

8. The composition of claim 7, wherein the peptide linker comprises 2-10 amino acid residues.

9. The composition of claim 1, wherein the molecular linker is a chemical linker.

10. The composition of claim 1, wherein the peptide is selected from the group of peptide fragments or variants that inhibit HIV viral entry consisting of the amino acid sequences:
(SEQ. ID. No. 2)
NYTSLIHSLIEESQNQQEKNEQELLELDKWASLWNWF;
(SEQ. ID. No. 3)
KYISLIHSLIEESQNQQEKNEQELLELDKWASLWNWF;
(SEQ. ID. No. 4)
HTTWMEWDREINNYTSLIHSLIEESQNQQEKNEQELL;
and
(SEQ. ID. No. 5)
HTTWMEWDREINKYISLIHSLIEESQNQQEKNEQELL.

11. A method of enhancing the anti-HIV potency of a given HIV C-peptide inhibitor, the method comprising physically joining a plurality of molecules of said C-peptide inhibitor by a molecular linker.

12. The method of claim 11, wherein two said C-peptide inhibitors are joined, forming a dimeric C-peptide inhibitor.

13. The method of claim 11, wherein three said C-peptide inhibitors are joined, forming a trimeric C-peptide inhibitor.

14. The method of claim 11, wherein the molecular linker is a peptide linker molecule.

15. The method of claim 11, wherein the molecular linker comprises at least 2 amino acids residues.

16. The method of claim 11, wherein the molecular linker is a chemical linker.

17. The method of claim 11, wherein the C-peptide inhibitor is selected from the group of C-peptides consisting of the amino acid sequences:
(SEQ. ID. No. 2)
NYTSLIHSLIEESQNQQEKNEQELLELDKWASLWNWF;
(SEQ. ID. No. 3)
KYISLIHSLIEESQNQQEKNEQELLELDKWASLWNWF;
(SEQ. ID. No. 4)
HTTWMEWDREINNYTSLIHSLIEESQNQQEKNEQELL;
(SEQ. ID. No. 5)
HTTWMEWDREINKYISLIHSLIEESQNQQEKNEQELL;
and
(SEQ. ID. No. 6)
WQEWEQKITALLEQAQIQQEKNEYELQKLDKWASLWEWF.

18. A pharmaceutical composition comprising a composition according to claim 1 and a pharmaceutically acceptable carrier.

19. An isolated nucleic acid that encodes a protein comprising a plurality of peptide having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein said plurality of peptides are physically joined by a peptide linker.

20. The isolated nucleic acid of claim 19, wherein the encoded protein is a dimer of two said peptides.

21. The isolated nucleic acid of claim 19, wherein the encoded protein is a trimer of three said peptides.

22. The isolated nucleic acid of claim 19, wherein the peptide linker molecule comprises at least 2 amino acids residues.

23. A method of treating HIV infection that is resistant to anti-HIV therapy in a subject, the method comprising administering to a subject in need thereof, an effective amount of a composition with anti-HIV activity, said composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein said peptides are physically joined by a molecular linker.

24. A method of preventing the development of HIV resistance to anti-HIV therapy in a subject, comprising administering to a subject in need thereof, an effective amount of a composition with anti-HIV activity, said composition comprising a plurality of peptides having the amino acid sequence HTTWMEWDREINNYTSLIHSLIEESQNQQEKNEQELLELDKWASLWNWF (SEQ. ID. No. 1) or fragments and/or variants thereof that inhibit HIV viral entry, wherein said peptides are physically joined by a molecular linker, in combination with an anti-HIV therapy, wherein the development of resistance is prevented.
